(19) United States
(12) Patent Application Publication
(10) Pub. No.: US 2006/0258628 A1
(43) Pub. Date: Nov. 16, 2006

(54) Compositions comprising 5-alpha reductase inhibitors, and SARMs and methods of use thereof

(76) Inventors: Mitchell S. Steiner, Germantown, TN (US); Karen A. Veverka, Cordova, TN (US); Duane D. Miller, Germantown, TN (US)

Correspondence Address: PEARL COHEN ZEDEK, LLP, 1500 BROADWAY 12TH FLOOR, NEW YORK, NY, 10036, US

(21) Appl. No.: 11/184,037

(22) Filed: Jul. 20, 2004

(51) Int. Cl. A61K 31/555; A61K 31/44; A61K 31/4172; A61K 31/506; A61K 31/415; A61K 31/32; A61K 31/277; A61K 31/165; A61K 31/66

(52) U.S. Cl. 514/114; 514/184; 514/256; 514/357; 514/400; 514/521; 514/616; 514/493; 514/514

(57) ABSTRACT

This invention provides a composition comprising a 5-alpha reductase inhibitor and a class of androgen receptor targeting agents (ARTA), which are selective androgen receptor modulators (SARM). The compositions of this invention are useful for, inter-alia, a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment of polycystic ovarian disease; e) treatment and/or prevention of acute and/or chronic muscular wasting conditions; f) preventing and/or treating dry eye conditions; g) oral androgen replacement therapy; h) decreasing the incidence of, halting or causing a regression of prostate cancer; and/or i) inducing apoptosis in a cancer cell.

### CROSS REFERENCE TO RELATED APPLICATIONS

This Application claims priority of U.S. Ser. No. 10/084,680, filed Feb. 28, 2003, and U.S. Ser. No. 10/371,209, filed Feb. 24, 2003, which are hereby incorporated by reference.

### FIELD OF INVENTION

The present invention relates to combinations of androgen receptor targeting agents (ARTA), which demonstrate antiandrogenic activity of a nonsteroidal ligand for the androgen receptor, and/or which bind irreversibly to the androgen receptor and 5-alpha reductase inhibitors. The combinations are useful for a) male contraception; b) treatment of a variety of hormone-related conditions, for example conditions associated with Androgen Decline in Aging Male (ADAM); c) treatment of conditions associated with Androgen Decline in Female (ADIF); d) treatment of polycystic ovarian disease e) treatment and/or prevention of acute and/or chronic muscular wasting conditions; f) preventing and/or treating dry eye conditions; g) oral androgen replacement therapy; h) decreasing the incidence of, halting or causing a regression of prostate cancer; and/or i) inducing apoptosis in a cancer cell.

### BACKGROUND OF THE INVENTION

The androgen receptor (“AR”) is a ligand-activated transcriptional regulatory protein that mediates induction of male sexual development and function through its activity with endogenous androgens. Androgens are generally known as the male sex hormones. The androgenic hormones are steroids, which are produced in the body by the testes and the cortex of the adrenal gland or can be synthesized in the laboratory. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Testosterone is the principal steroid secreted by the testes and is the primary circulating androgen found in the plasma of males. Testosterone is converted to DHT by the enzyme 5 alpha-reductase in many peripheral tissues. DHT is thus thought to serve as the intracellular mediator for most androgen actions (Zhou, et al., Molec. Endocrinol. 9:208-18 (1995)). Other steroidal androgens include esters of testosterone, such as the cypionate, propionate, phenylpropionate, cyclopentylpropionate, isocarporate, enanthate, and decanoate esters, and other synthetic androgens such as 7-Methyl-Nortestosterone (“MENT”) and its acetate ester (Sundaram et al., “7 Alpha-Methyl-Nortestosterone(MENT): The Optimal Androgen For Male Contraception,” Ann. Med., 25:199-205 (1993) (“Sundaram”)). Because the AR is involved in male sexual development and function, the AR is a likely target for effecting male contraception or other forms of hormone replacement therapy.

Worldwide population growth and social awareness of family planning have stimulated a great deal of research in contraception. Contraception is a difficult subject under any circumstance. It is fraught with cultural and social stigma, religious implications, and, most certainly, significant health concerns. This situation is only exacerbated when the subject focuses on male contraception. Despite the availability of suitable contraceptive devices, historically, society has looked to women to be responsible for contraceptive decisions and their consequences. Although concern over sexually transmitted diseases has made men more aware of the need to develop safe and responsible sexual habits, women still often bear the brunt of contraceptive choice. Women have a number of choices, from temporary mechanical devices such as sponges and diaphragms to temporary chemical devices such as spermicides. Women also have at their disposal more permanent options, such as physical devices including IUDs and cervical caps as well as more permanent chemical treatments such as birth control pills and subcutaneous implants. However, to date, the only options available for men include the use of condoms and vasectomy. Condom use, however is not favored by many men because of the reduced sexual sensitivity, the interruption in sexual spontaneity, and the significant possibility of pregnancy caused by breakage or misuse. Vasectomies are also not favored. If more convenient methods of birth control were available to men, particularly long-term methods which require no preparative activity immediately prior to a sexual act, such methods could significantly increase the likelihood that men would take more responsibility for contraception.

Administration of the male sex steroids (e.g., testosterone and its derivatives) has shown particular promise in this regard due to the combined gonadotropin-suppressing and androgen-substituting properties of these compounds (Steinberger et al., “Effect of Chronic Administration of Testosterone Enanthate on Sperm Production and Plasma Testosterone, Follicle Stimulating Hormone, and Luteinizing Hormone Levels: A Preliminary Evaluation of a Possible Male Contraceptive, Fertility and Sterility 28:1320-28 (1977)). Chronic administration of high doses of testosterone completely abolishes sperm production (azoospermia) or reduces it to a very low level (oligospermia). The degree of spermatogenic suppression necessary to produce infertility is not precisely known. However, a recent report by the World Health Organization showed that weekly intramuscular injections of testosterone enanthate result in azoospermia or severe oligospermia (i.e., less than 3 million sperm per ml) and infertility in 98% of men receiving therapy (World Health Organization Task Force on Methods And Regulation of Male Fertility, “Contraceptive Efficacy of Testosterone-Induced Azoospermia and Oligospermia in Normal Men,” Fertility and Sterility 65:821-29 (1996)).

A variety of testosterone esters have been developed which are more slowly absorbed after intramuscular injection and thus result in greater androgenic effect Testosterone enanthate is the most widely used of these esters. While testosterone enanthate has been valuable in terms of establishing the feasibility of hormonal agents for male contraception, it has several drawbacks, including the need for weekly injections and the presence of supraphysiologic peak levels of testosterone immediately following intramuscular injection (Wu, “Effects of Testosterone Enanthate in Normal Men: Experience From a Multicenter Contraceptive Efficacy Study,” Fertility and Sterility 65:626-36 (1996)).

Steroidal ligands which bind the AR and act as androgens (e.g. testosterone enanthate) or as antiandrogens (e.g. cyproterone acetate) have been known for many years and are used clinically (Wu 1988). Although nonsteroidal antiandrogens are in clinical use for hormone-dependent prostate cancer, nonsteroidal androgens have not been reported. For this reason, research on male contraceptives has focused solely on steroidal compounds.

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients. Another strategy, however, is to develop drugs to prevent prostate cancer. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer. Thus, the development of treatment and preventative strategies against prostate cancer may have the greatest overall impact both medically and economically against prostate cancer.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated. The elderly are at greatest risk of osteoporosis, and the problem is therefore predicted to increase significantly with the aging of the population. Worldwide fracture incidence is forecasted to increase three-fold over the next 60 years, and one study estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake. However, osteoporosis also occurs frequently in males. It is well established that the bone mineral density of males decrease with age. Decreased amounts of bone mineral content and density correlates with decreased bone strength, and predisposes to fracture. The molecular mechanisms underlying the pleiotropic effects of sex-hormones in non-reproductive tissues are only beginning to be understood, but it is clear that physiologic concentrations of androgens and estrogens play an important role in maintaining bone homeostasis throughout the life-cycle. Consequently, when androgen or estrogen deprivation occurs there is a resultant increase in the rate of bone remodeling that tilts the balance of resorption and formation to the favor of resorption that contributes to the overall loss of bone mass. In males, the natural decline in sex-hormones at maturity (direct decline in androgens as well as lower levels of estrogens derived from peripheral aromatization of androgens) is associated with the frailty of bones. This effect is also observed in males who have been castrated.

Polycystic Ovarian Syndrome (PCOS) is characterized by menstrual irregularity and hirsutism and is a common cause of anovulatory infertility. The biochemical abnormalities are a high concentration of plasma luteinising hormone (LH) or a high LH/follicle stimulating hormone (FSH) ratio and high concentrations of androgens (testosterone and/or androstenedione and/or dehydroepiandrosterone (DHEA). Excess androgen secretion is by the ovary and/or the adrenal gland. Clinical manifestations of PCOS include amenorrhea, hirsutism acanthosis nigricans, acne and obesity. Women with PCOS are typically hirsute, infertile, and present with an increased risk, and early onset of diabetes and cardiovascular disease.

Androgen decline in the aging male (ADAM) refers to a progressive decrease in androgen production, common in males after middle age. The syndrome is characterized by alterations in the physical and intellectual domains that correlate with and can be corrected by manipulation of the androgen milieu. ADAM is characterized biochemically by a decrease not only in serum androgen, but also in other hormones, such as growth hormone, melatonin and dehydroepiandrosterone. Clinical manifestations include fatigue, depression, decreased libido, sexual dysfunction, erectile dysfunction, hypogonadism, osteoporosis, hair loss, obesity, sarcopenia, osteopenia, benign prostate hyperplasia, anemia, alterations in mood and cognition and prostate cancer.

Androgen Deficiency in Female (ADIF) refers to a variety of hormone-related conditions including, common in females after middle agest. The syndrome is characterized by sexual dysfunction, decreased sexual libido, hypogonadism, sarcopenia, osteopenia, osteoporosis, alterations in cognition and mood, anemia, depression, anemia, hair loss, obesity, endometriosis, breast cancer, uterine cancer and ovarian cancer.

Muscle wasting refers to the progressive loss of muscle mass and/or to the progressive weakening and degeneration of muscles, including the skeletal or voluntary muscles, which control movement, cardiac muscles, which control the heart (cardiomyopathics), and smooth muscles. Chronic muscle wasting is a chronic condition (i.e. persisting over a long period of time) characterized by progressive loss of muscle mass, weakening and degeneration of muscle The loss of muscle mass that occurs during muscle wasting can be characterized by a muscle protein breakdown or degradation. Protein degradation occurs because of an unusually high rate of protein degradation, an unusually low rate of protein synthesis, or a combination of both. Protein degradation, whether caused by a high degree of protein degradation or a low degree of protein synthesis, leads to a decrease in muscle mass and to muscle wasting. Muscle wasting is associated with chronic, neurological, genetic or infectious pathologies, diseases, illnesses or conditions. These include Muscular Dystrophies such as Duchenne Muscular Dystrophy and Myotonic Dystrophy; Muscle Atrophies such as Post-Polio Muscle Atrophy (PPMA); Cachexias such as Cardiac Cachexia, AIDS Cachexia and Cancer Cachexia, malnutrition, Leprosy, Diabetes, Renal Disease, Chronic Obstructive Pulmonary Disease (COPD), Cancer, end stage Renal failure, Emphysema, Osteomalacia, HIV Infection, AIDS, and Cardiomyopathy, In addition, other circumstances and conditions are linked to and can cause muscle wasting. These include chronic lower back pain, advanced age, central nervous system (CNS) injury, peripheral nerve injury, spinal cord injury, chemical injury, central nervous system (CNS) damage, peripheral nerve damage, spinal cord damage, chemical damage, burns, disuse deconditioning that occurs when a limb is immobilized, long term hospitalization due to illness or injury, and alcoholism. Muscle wasting, if left unabated, can have dire health consequences. For example, the changes that occur during muscle wasting can lead to a weakened physical state that is detrimental to an individual's health, resulting in increased susceptibility to infection, poor performance status and susceptibility to injury.

New innovative approaches are urgently needed at both the basic science and clinical levels to develop compounds which are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with ADIF, such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment of polycystic ovarian syndrome; e) treatment and/or prevention of acute and/or chronic muscular wasting conditions; f) preventing and/or treating dry eye conditions; g) oral androgen replacement therapy; h) decreasing the incidence of halting or causing a regression of prostate cancer; and/or i) inducing apoptosis in a cancer cell.

### SUMMARY OF THE INVENTION

In one embodiment, this invention provides a composition comprising a 5-alpha reductase inhibitor and an androgen receptor targeting agent (ARTA), which is a selective androgen receptor modulator (SARM). In one embodiment, the SARM compounds bind irreversibly to the androgen receptor. In another embodiment, the SARM compounds are androgen receptor antagonists, which bind irreversibly to the androgen receptor. In another embodiment, the SARM compounds are alkylating agents.

The compositions are useful, in some embodiments, for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment of polycystic ovarian syndrome; e) treatment and/or prevention of acute and/or chronic muscular wasting conditions; f) preventing and/or treating dry eye conditions; g) oral androgen replacement therapy; h) decreasing the incidence of, halting or causing a regression of prostate cancer; and/or i) inducing apoptosis in a cancer cell.

In one embodiment, the present invention provides a composition comprising a 5-alpha reductase inhibitor and a selective androgen receptor modulator (SARM) compound represented by the structure of formula I: 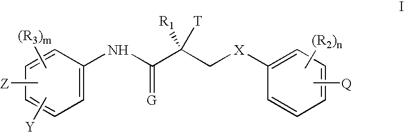
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 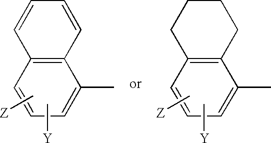
Z is NO₂, CN, COR, COOH, or CONHR;
Y is CF₃, F, Br, CL L CN, or SnR₃;
Q is SCN, NCS, OCN, or NCO;
n is an integer of 1-4;
m is an integer of 1-3; and
wherein all unspecified positions can be substituted or unsubstituted.

In another embodiment, the composition will comprise a 5-alpha reductase inhibitor and a SARM compound, which is an analog, derivative, isomer, metabolite, pharmaceutically acceptable salt, pharmaceutical product, hydrate or N-oxide of the compound of formula I, or any combination thereof.

In one embodiment, G in the compound of formula I is O. In another embodiment, X in the compound of formula I is O. In another embodiment, T in the compound of formula I is OH. In another embodiment, R₁ the compound of formula I is CH₃. In another embodiment, Z the compound of formula I is NO₂. In another embodiment, Z in the compound of formula I is CN. In another embodiment, Y in the compound of formula I is CF₃. In another embodiment, Q in the compound of formula I is NCS. In another embodiment, Q in the compound of formula I is in the para position. In another embodiment, Z in the compound of formula I is in the para position. In another embodiment, Y in the compound of formula I is in the meta position. In another embodiment, G in the compound of formula I is O, T is OH, R₁ is CH₃, X is O, Z is NO₂, Y is CF₃, and Q is NCS.

In another embodiment, the present invention provides a composition comprising a 5-alpha reductase inhibitor and a selective androgen receptor modulator (SARM) compound represented by the structure of formula II:

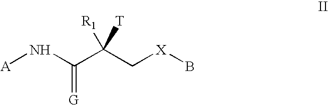

wherein
X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 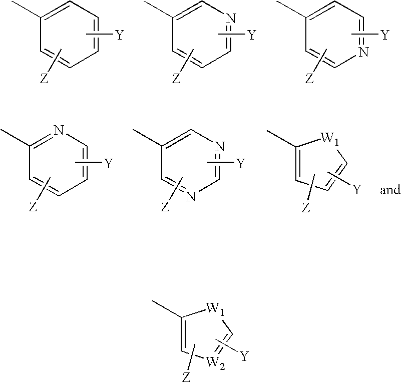
B is a ring selected from:

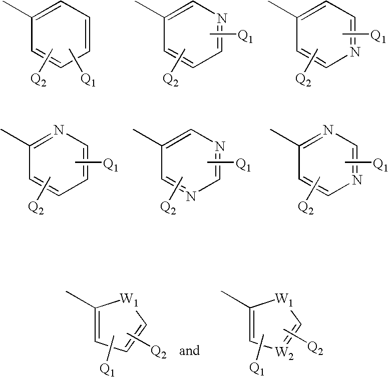

wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 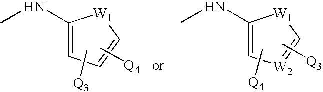
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO; and
wherein all unspecified positions can be substituted or unsubstituted.

In another embodiment, the composition will comprise a 5-alpha reductase inhibitor and a SARM compound, which is an an analog, derivative, isomer, metabolite, pharmaceutically acceptable salt, pharmaceutical product, hydrate or N-oxide of the compound of formula II, or any combination thereof.

In one embodiment, G the compound of formula II is O. In another embodiment, X in the compound of formula II is O. In another embodiment, T in the compound of formula II is OH. In another embodiment, R₁ in the compound of formula II is CH₃. In another embodiment, Z in the compound of formula II is NO₂. In another embodiment, Z in the compound of formula II is CN. In another embodiment, Y in the compound of formula II is CF₃. In another embodiment, Q₁ in the compound of formula II is NCS. In another embodiment, Q₁ in the compound of formula II is in the para position. In another embodiment, Z in the compound of formula II is in the para position. In another embodiment, Y in the compound of formula II is in the meta position. In another embodiment, G in the compound of formula II is O, T is OH, R₁ is CH₃, X is O, Z is NO₂, Y is CF₃, and Q₁ is NCS.

In another embodiment, the present invention provides a composition comprising a 5-alpha reductase inhibitor and a selective androgen receptor modulator (SARM) compound represented by the structure of formula III:

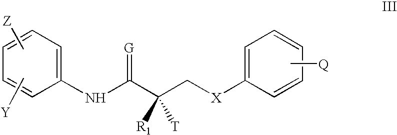

wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR,
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In another embodiment, the composition will comprise a 5-alpha reductase inhibitor and a SARM compound, which is an analog, derivative, isomer, metabolite, pharmaceutically acceptable salt, pharmaceutical product, hydrate or N-oxide of the compound of formula III, or any combination thereof.

In one embodiment, G in the compound of formula III is O. In another embodiment, X in the compound of formula III is O. In another embodiment, T in the compound of formula III is OH. In another embodiment, R₁ in the compound of formula III is CH₃. In another embodiment, Z in the compound of formula III is NO₂. In another embodiment, Z in the compound of formula III is CN. In another embodiment, Y in the compound of formula III is CF₃. In another embodiment, Q in the compound of formula III is NCS. In another embodiment, Q in the compound of formula III is in the para position. In another embodiment, Z in the compound of formula III is in the para position. In another embodiment, Y in the compound of formula III is in the meta position. In another embodiment, G in the compound of formula III is O, T is OH, R₁ is CH₃, X is O, Z is NO₂, Y is CF₃, and Q is NCS.

In another embodiment, the present invention provides a composition comprising a 5-alpha reductase inhibitor and a selective androgen receptor modulator (SARM) compound represented by the structure of formula IV:

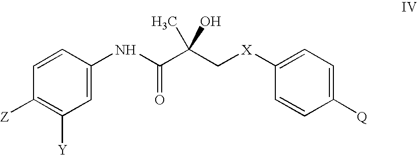

wherein
X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

In another embodiment, the composition will comprise a 5-alpha reductase inhibitor and a SARM compound, which is an analog, derivative, isomer, metabolite, pharmaceutically acceptable salt, pharmaceutical product, hydrate or N-oxide of the compound of formula IV, or any combination thereof.

In one embodiment, X in the compound of formula IV is O. In another embodiment, Z in the compound of formula IV is NO₂. In another embodiment, Z in the compound of formula IV is CN. In another embodiment, Y in the compound of formula IV is CF₃. In another embodiment, Q in the compound of formula IV is NCS.

In another embodiment, the present invention provides a composition comprising a 5-alpha reductase inhibitor and a selective androgen receptor modulator (SARM) compound represented by the structure of formula V, and/or an analog, derivative, isomer, metabolite, pharmaceutically acceptable salt, pharmaceutical product, hydrate, N-oxide, or any combination thereof.

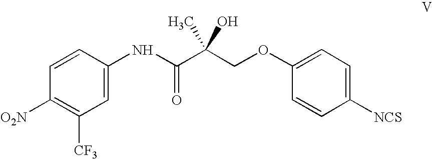

In one embodiment, the SARM compound of any of formulas I-V is an androgen receptor antagonist. In another embodiment, the SARM compound of any of formulas I-V binds irreversibly to an androgen receptor. In another embodiment, the SARM compound of any of formulas I-V is an androgen receptor antagonist, which binds irreversibly to an androgen receptor. In another embodiment, the SARM compound of any of formulas I-V is an alkylating agent.

In another embodiment, the 5-alpha reductase inhibitor is dutasteride or finasteride, or a combination thereof. In another embodiment, the composition comprises a suitable carrier or diluent.

In another embodiment, the present invention provides a method of suppressing spermatogenesis in a subject, comprising the step of administering a composition of this invention to the subject, in an amount effective to suppress sperm production, in an amount effective to suppress sperm production.

In another embodiment, the present invention provides a method of contraception in a male subject, comprising the step of administering a composition of this invention to the subject, in an amount effective to suppress sperm production in the subject, thereby effecting contraception in the subject.

In another embodiment, the present invention further provides a method of hormone therapy, comprising the step of administering a composition of this invention to the subject, in an amount effective to effect a change in an androgen-dependent condition.

In another embodiment, the present invention provides a method of hormone replacement therapy comprising administering a composition of this invention to the subject, in an amount effective to effect a change in an androgen-dependent condition.

In another embodiment, the present invention further provides a method of treating a subject suffering from prostate cancer, comprising the step of administering a composition of this invention to the subject, in an amount effective to treat prostate cancer in the subject.

In another embodiment, the present invention provides a method of suppressing, inhibiting or preventing prostate cancer, or its relapse in a subject, comprising the step of administering a composition of this invention to the subject, in an amount effective to suppress, inhibit or prevent prostate cancer or its relapse in the subject.

In another embodiment, the present invention further provides a method of preventing the recurrence of prostate cancer in a subject suffering from prostate cancer, comprising the step of administering a composition of this invention to the subject, in an amount effective to prevent the recurrence of prostate cancer in the subject.

In another embodiment, the present invention further provides a method of treating or preventing benign prostatic hyperplasia, comprising the step of administering a composition of this invention to the subject, in an amount effective to treat or prevent benign prostatic hyperplasia.

In another embodiment, the present invention provides a method of treating or preventing a dry eye condition in a subject suffering from dry eyes, comprising the step of administering a composition of this invention to the subject, in an amount effective to treat or prevent dry eyes in the subject.

In another embodiment, the present invention provides a method of treating polycystic ovarian syndrome in a female subject, comprising the step of administering a composition of this invention to the subject, in an amount effective to treat polycystic ovarian syndrome.

In another embodiment, the present invention provides a method of suppressing, inhibiting, delaying onset or preventing diabetes, breast cancer, endometrial carcinoma or cardiovascular disease in a female subject suffereing from polycystic ovarian syndrome, comprising the step of administering a composition of this invention to the subject, in an amount effective to suppress, inhibit, delay onset, or prevent diabetes, breast cacner, endometrial carcinoma or cardiovascular disease in the subject.

### DETAILED DESCRIPTION OF THE INVENTION

In one embodiment, this invention provides a composition comprising a 5-alpha reductase inhibitor and an androgen receptor targeting agent (ARTA), which is a selective androgen receptor modulator (SARM). In one embodiment, the SARM compounds bind irreversibly to the androgen receptor. In another embodiment, the SARM compounds are androgen receptor antagonists which bind irreversibly to the androgen receptor. In another embodiment, the SARM compounds are alkylating agents.

The compositions of this invention are useful, inter-alia, for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment of polycystic ovarian syndromes and diseases associated with the syndrome; e) treatment and/or prevention of acute and/or chronic muscular wasting conditions; f) preventing and/or treating dry eye conditions; g) oral androgen replacement therapy; h) decreasing the incidence of, halting or causing a regression of prostate cancer; and/or i) inducing apoptosis in a cancer cell.

In one embodiment, the present invention provides a composition comprising a 5-alpha reductase inhibitor and a selective androgen receptor modulator (SARM) compound represented by the structure of formula I: 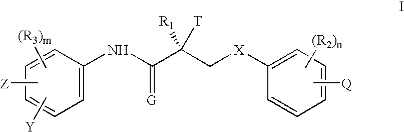
X is a bond, O, CH₂, NH, S, Se, PR, NO or NR,
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, L CN, NO₂, COR, COOH, CONHR, CF₃, SnR₃, or R₃ together with the benzene ring to which it is attached forms a fused ring system represented by the structure: 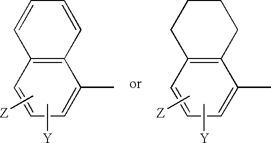
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

In one embodiment, this invention provides a composition comprising a 5-alpha reductase inhibitor and a SARM compound, which is an analog of the compound of formula I, or in another embodiment, a derivative of the compound of formula I, or in another embodiment, an isomer of the compound of formula I, or in another embodiment, a metabolite of the compound of formula I, or in another embodiment, a pharmaceutically acceptable salt of the compound of formula I, or in another embodiment, a pharmaceutical product of the compound of formula I, or in another embodiment, a hydrate of the compound of formula I, or in another embodiment, an N-oxide of the compound of formula I, or in another embodiment, a combination of any of an analog, derivative, metabolite, isomer, pharmaceutically acceptable salt, pharmaceutical product, hydrate or N-oxide of the compound of formula I.

In one embodiment, G in the compound of formula I is O. In another embodiment, X in the compound of formula I is O. In another embodiment, T in the compound of formula I is OH. In another embodiment, R₁ the compound of formula I is CH₃. In another embodiment, Z the compound of formula I is NO₂. In another embodiment, Z in the compound of formula I is CN. In another embodiment, Y in the compound of formula I is CF₃. In another embodiment, Q in the compound of formula I is NCS. In another embodiment, Q in the compound of formula I is in the para position. In another embodiment, Z in the compound of formula I is in the para position. In another embodiment, Y in the compound of formula I is in the meta position. In another embodiment, G in the compound of formula I is O, T is OH, R₁ is CH₃, X is O, Z is NO₂, Y is CF₃, and Q is NCS.

In another embodiment, the present invention provides a composition comprising a 5-alpha reductase inhibitor and a selective androgen receptor modulator (SARM) compound represented by the structure of formula II:

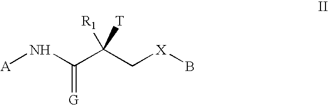

wherein
X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 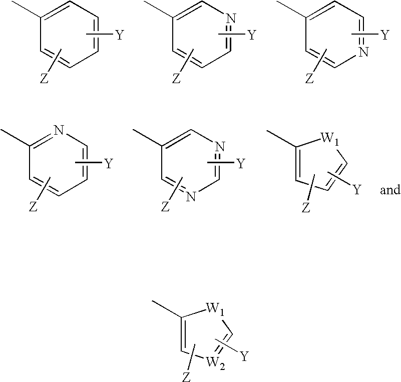
B is a ring selected from:

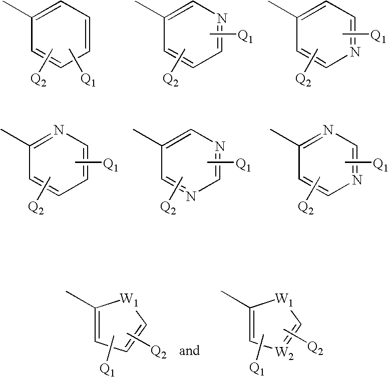

wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR. NHSO₂CH₃, NHSO₂R, OR, COR, OCOR, OSO₂R, SO₂R, SR, 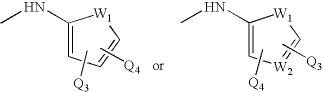
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO; and
wherein all unspecified positions can be substituted or unsubstituted.

In another embodiment, the composition will comprise a 5-alpha reductase inhibitor and a SARM compound, which is an an analog, derivative, isomer, metabolite, pharmaceutically acceptable salt, pharmaceutical product, hydrate or N-oxide of the compound of formula II, or any combination thereof.

In one embodiment, G the compound of formula II is O. In another embodiment, X in the compound of formula II is O. In another embodiment, T in the compound of formula II is OH. In another embodiment, R₁ in the compound of formula II is CH₃. In another embodiment, Z in the compound of formula II is NO₂. In another embodiment, Z in the compound of formula II is CN. In another embodiment, Y in the compound of formula II is CF₃. In another embodiment, Q₁ in the compound of formula II is NCS. In another embodiment, Q₁ in the compound of formula II is in the para position. In another embodiment, Z in the compound of formula II is in the para position. In another embodiment, Y in the compound of formula II is in the meta position. In another embodiment, G in the compound of formula II is O, T is OH, R₁ is CH₃, X is O, Z is NO₂, Y is CF₃, and Q, is NCS.

In another embodiment, the present invention provides a composition comprising a 5-alpha reductase inhibitor and a selective androgen receptor modulator (SARM) compound represented by the structure of formula III:

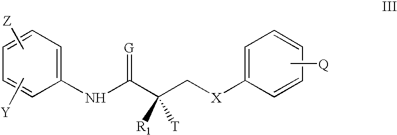

wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In another embodiment, the composition will comprise a 5-alpha reductase inhibitor and a SARM compound, which is an analog, derivative, isomer, metabolite, pharmaceutically acceptable salt, pharmaceutical product, hydrate or N-oxide of the compound of formula III, or any combination thereof.

In one embodiment, G in the compound of formula III is O. In another embodiment, X in the compound of formula III is O. In another embodiment, T in the compound of formula III is OH. In another embodiment, R₁ in the compound of formula III is CH₃. In another embodiment, Z in the compound of formula III is NO₂. In another embodiment, Z in the compound of formula III is CN. In another embodiment, Y in the compound of formula III is CF₃. In another embodiment, Q in the compound of formula III is NCS. In another embodiment, Q in the compound of formula III is in the para position. In another embodiment, Z in the compound of formula III is in the para position. In another embodiment, Y in the compound of formula III is in the meta position. In another embodiment, G in the compound of formula III is O, T is OH, R₁ is CH₃, X is O, Z is NO₂, Y is CF₃, and Q is NCS.

In another embodiment, the present invention provides a composition comprising a 5-alpha reductase inhibitor and a selective androgen receptor modulator (SARM) compound represented by the structure of formula IV:

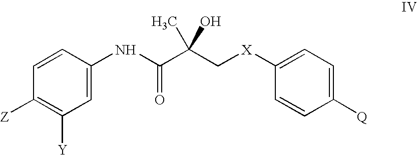

wherein
X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is allyl, haloalkyl, dihaloalkyl, trihaloalkyl, CH₂F, CHF₂, CF₃, CF₂CF₃, aryl, phenyl, halogen, alkenyl or OH.

In another embodiment, the composition will comprise a 5-alpha reductase inhibitor and a SARM compound, which is an analog, derivative, isomer, metabolite, pharmaceutically acceptable salt, pharmaceutical product, hydrate or N-oxide of the compound of formula IV, or any combination thereof.

In one embodiment, X in the compound of formula IV is O. In another embodiment, Z in the compound of formula IV is NO₂. In another embodiment, Z in the compound of formula IV is CN. In another embodiment, Y in the compound of formula IV is CF₃. In another embodiment, Q in the compound of formula IV is NCS.

In another embodiment, the present invention provides a composition comprising a 5-alpha reductase inhibitor and a selective androgen receptor modulator (SARM) compound represented by the structure of formula V, and/or an analog, derivative, isomer, metabolite, pharmaceutically acceptable salt, pharmaceutical product, hydrate, N-oxide, or any combination thereof.

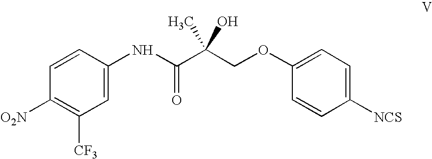

As contemplated herein, other specific embodiments of SARM compounds included within the scope of the present invention are compounds VI and VII. It is understood that included within the scope of the present invention are analogs, derivatives, metabolites, isomers, pharmaceutically acceptable salts, pharmaceutical products, hydrates, N-oxides or combinations thereof of these compounds:

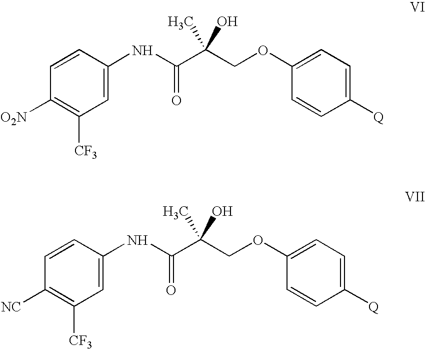

wherein Q is NCS, SCN, NCO or OCN.

The substituent R is defined herein as an alkyl, haloalkyl, dihaloalkyl, trihaloalkyl, CH₂F, CHF₂, CF₃, CF₂CF₃; aryl, phenyl, halogen, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I, each of which represents an embodiment of this invention.

An “aryl” group refers, in one embodiment, to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond. A halo group refers, in other embodiments, to F, Cl, Br or I.

An “arylalkyl” group refers to an alkyl bound to an aryl, wherein alkyl and aryl are as defined above. An example of an aralkyl group is a benzyl group.

It is to be understood, with regard to the SARM compounds utilized in the compositions of this invention, that any SARM compound which corresponds to a compound of the formulas presented herein may be utilized, and that any unspecified position in the formulas may be substituted or unsubstituted.

As contemplated herein, the present invention relates to the use combinations of a 5-alpha reductase inhibitor and a SARM compound and/or its analog, derivative, isomer, metabolite, pharmaceutically acceptable salt, pharmaceutical product, hydrate, N-oxide, or combinations thereof.

In one embodiment, the term “isomer” includes, but is not limited to, optical isomers and analogs, structural isomers and analogs, conformational isomers and analogs, and the like.

In one embodiment, this invention encompasses the use of various optical isomers of the SARM compound. It will be appreciated by those skilled in the art that the SARMs of the present invention contain at least one chiral center. Accordingly, the SARMs used in the compositions and methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of androgen-related conditions described herein. In one embodiment, the SARMs are the pure (R)-isomers. In another embodiment, the SARMs are the pure (S)-isomers. In another embodiment, the SARMs are a mixture of the (R) and the (S) isomers. In another embodiment, the SARMs are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the SARM compounds. The term “derivatives” includes but is not limited to ether derivatives, acid derivatives, amide derivatives, ester derivatives and the like. In addition, this invention further includes hydrates of the SARM compounds. The term “hydrate” includes but is not limited to hemihydrate, monohydrate, dihydrate, trihydrate and the like.

This invention further includes metabolites of the SARM compounds. The term “metabolite” means any substance produced from another substance by metabolism or a metabolic process.

This invention further includes pharmaceutical products of the SARM compounds. The term “pharmaceutical product” means a composition suitable for pharmaceutical use (pharmaceutical composition), as defined herein.

In another embodiment, the present invention provides process for preparing the compositions comprising at least one 5-alpha reductase inhibitor and at least one selective androgen receptor modulator (SARM) compounds of the present invention, which may include methods for the synthesis of non-steroidal agonist compounds, that can be used for industrial large-scale synthesis, and provide highly pure products in high yield.

In one embodiment, the process for preparing the selective androgen receptor modulator (SARM) compound represented by the structure of formula I:

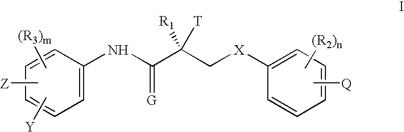

wherein
X is a O, NH, S, Se, PR, or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, N NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 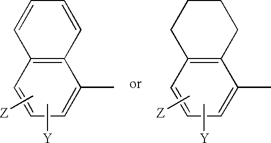
Z is NO₂, CN, COR, COOH, or CONHR,
Y is CF₃, F, Br, Cl, I, CN, or SnR₃;
Q is SCN, NCS, OCN, or NCO;
n is an integer of 1-4; and
m is an integer of 1-3;

may comprise the step of coupling a compound of formula VIII:

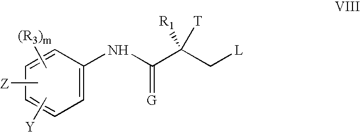

wherein Z, Y, G, R₁, T, R₃ and m are as defined above and L is a leaving group,

with a compound of formula IX:

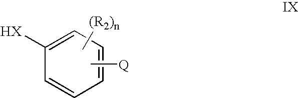

wherein Q, X R₂ and n are as defined above.

In one embodiment, the coupling step is carried out in the presence of a base. In another embodiment, the leaving group L is Br. In another embodiment, the compound of formula VIII is prepared by
a) preparing a compound of formula X by ring opening of a cyclic compound of formula XI 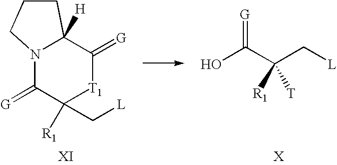
wherein L, R₁, G and T are as defined above, and T₁ is O or NH; and
b) reacting an amine of formula XII:

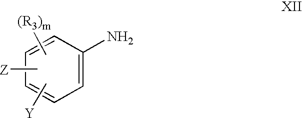

wherein Z, Y, R₃ and m are as defined above, with the compound of formula X, in the presence of a coupling reagent, to produce the compound of formula VIII.

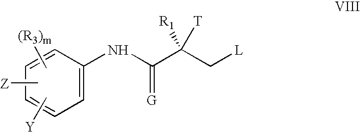

In one embodiment, step (a) is carried out in the presence of HBr. In another embodiment, the process further comprises the step of converting the selective androgen receptor modulator (SARM) compound to its analog, isomer, metabolite, derivative, pharmaceutically acceptable salt, pharmaceutical product, N-oxide, hydrate or any combination thereof.

In another embodiment, the process for preparing a selective androgen receptor modulator (SARM) compound represented by the structure of formula II:

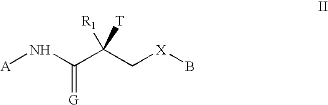

wherein
X is O, NH, S, Se, PR, or NR;
G is O or S;
R₁ is CH₃, CH₂F, CHF₃, CF₃, CH₂CH₃, or CF₂CF₃,
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 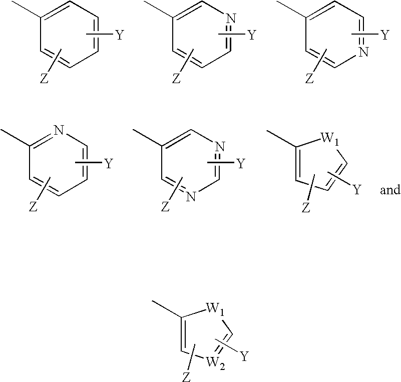
B is a ring selected from:

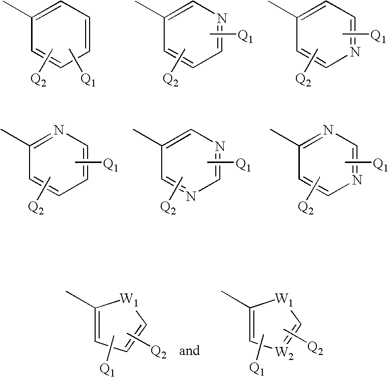

wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 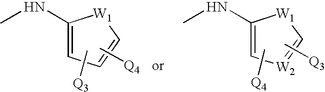
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;

May comprise the step of coupling a compound of formula III:

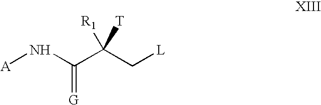

wherein A, G, R₁ and T are as defined above and L is a leaving group,

with a compound of formula HX-B wherein B and X are as defined above.

In one embodiment, the coupling step is carried out in the presence of a base. In another embodiment, the leaving group L is Br. In another embodiment, the compound of formula XIII is prepared by
a) preparing a compound formula X by ring opening of a cyclic compound of formula XI 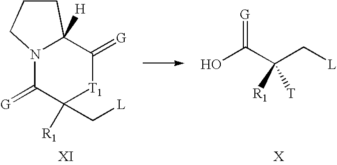
wherein L, R₁, G and T are as defined above, and T₁ is O or NH; and
b) reacting an amine of formula A-NH₂ wherein A is as defined above, with the compound of formula X in the presence of a coupling reagent, to produce the amide of formula III.

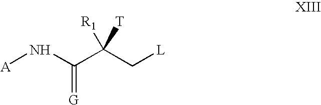

In one embodiment, step (a) is carried out in the presence of HBr. In another embodiment, the process further comprises the step of converting the selective androgen receptor modulator (SARM) compound to its analog, isomer, metabolite, derivative, pharmaceutically acceptable salt, pharmaceutical product, N-oxide, hydrate or any combination thereof.

In another embodiment, the process for preparing a selective androgen receptor modulator (SARM) compound represented by the structure of formula III:

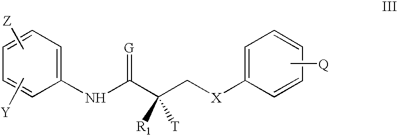

wherein
X is O, NH, S, Se, PR or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;

the process comprising the step of coupling a compound of formula XIV:

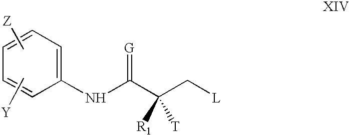

wherein Z, Y, G R₁ and T are as defined above and L is a leaving group,

with a compound of formula XV:

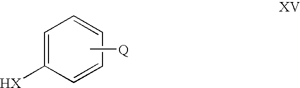

wherein Q and X are as defined above.

In one embodiment, the coupling step is carried out in the presence of a base. In another embodiment, the leaving group L is Br. In another embodiment, the compound of formula XIV is prepared by
a) preparing a compound formula X by ring opening of a cyclic compound of formula XI 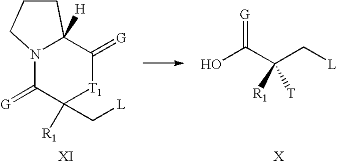
wherein L, R₁, and T are as defined above, G is O and T₁ is O or NH; and
b) reacting an amine of formula XVI

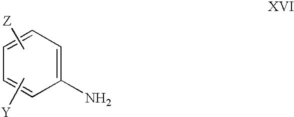

with the compound of formula X in the presence of a coupling reagent, to produce the compound of formula XIV.

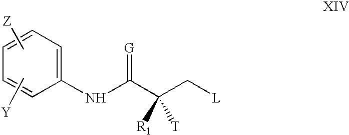

In one embodiment, step (a) is carried out in the presence of HBr. In another embodiment, the process further comprises the step of converting the selective androgen receptor modulator (SARM) compound to its analog, isomer, metabolite, derivative, pharmaceutically acceptable salt, pharmaceutical product, N-oxide, hydrate or any combination thereof.

In another embodiment, the process for preparing a selective androgen receptor modulator (SARM) compound represented by the structure of formula IV: 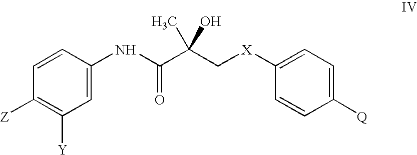
wherein X is O, NH, S, Se, PR, or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH;

may comprise the step of coupling an amide of formula XVII:

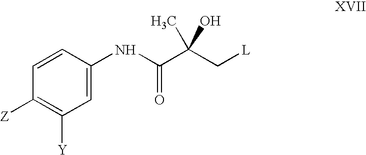

wherein Z and Y are as defined above and L is a leaving group, with a compound of formula XVIII

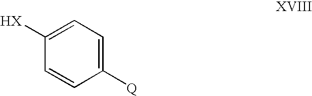

wherein Q and X R₂ are as defined above.

In one embodiment, the coupling step is carried out in the presence of a base. In another embodiment, the leaving group L is Br. In another embodiment, the compound of formula XVII is prepared by
a) preparing a compound formula X by ring opening of a cyclic compound of formula XI 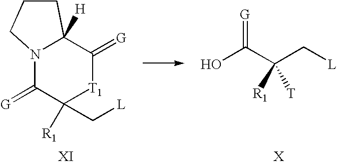
wherein L, R₁, and T are as defined above, G is O and T₁ is O or NH; and
b) reacting an amine of formula XVIX 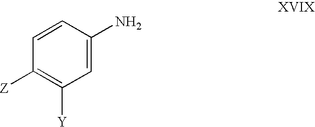
   with the compound of formula X in the presence of a coupling reagent, to produce the compound of formula XVII.

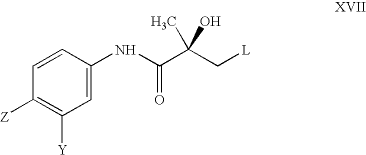

In one embodiment, step (a) is carried out in the presence of HBr. In another embodiment, the process further comprises the step of converting the selective androgen receptor modulator (SARM) compound to its analog, isomer, metabolite, derivative, pharmaceutically acceptable salt, pharmaceutical product, N-oxide, hydrate or any combination thereof.

As demonstrated herein, Applicants have found that when the purification step of the SARM compounds is carried out in the presence of a nontoxic organic solvent and water, such as ethanol and water, for example by recrystallization from a mixture of ethanol and water, a highly pure product with excellent crystal stability is obtained in high yields. In addition, the use of a nontoxic organic solvent/water for purification is safe and cheap, and avoids any biological hazards that may arise from the use of toxic organic solvents such as hexane. In one embodiment, the nontoxic organic solvent is ethanol.

Thus, in one embodiment, the process for preparing the SARM compounds described herein, may involve a purification step comprising crystallization of the SARM product using a mixture of a nontoxic organic solvent and water. In one embodiment, the nontoxic organic solvent is ethanol. In another embodiment, the crystallization step comprises mixing an ethanol solution comprising the SARM compound with water, so as to crystallize the SARM compound. In a further embodiment, the process further comprises the step of collecting the SARM compound by filtration.

The process may be, in another embodiment, for large-scale preparation, and providing highly pure products in high yield. The methods for producing a SARM compound for the compositions of this invention, may utilize safe, environmentally friendly and cheap reagents and purification steps, thus avoiding any undesirable toxicological issues that may arise from the use of toxic, environmentally unfriendly or biologically unstable reagents.

It should be apparent to a person skilled in the art that any nontoxic organic solvent is suitable in preparing the compositions of the present invention, for example alcohols such as methanol or ethanol, aromatic compounds such as toluene and xylene, DMSO, THF, cyclohexane and the like.

In one embodiment, the nontoxic organic solvent is ethanol. Any grade and purity level of ethanol is suitable. In one embodiment, the ethanol is neat ethanol. In another embodiment, the ethanol is an ethanol solution that contains denaturants, such as toluene, methanol and the like.

It is understood to ta person skilled in the art that when T₁ is O or NH, T is compound VIII is O or NH₂. Thus, when T in compound I is OR, the reaction will involve a further step of converting the OH to OR by a reaction with, for example, an alkyl halide R—X. When T in compound I is NHCOR, NHCOCH₃, the reaction will involve a further step of converting the NH₂ to NHCOR or NHCOCH₃, by a reaction with, for example, the corresponding acyl chloride ClCOR or ClCOCH₃.

In one embodiment, the coupling step defined hereinabove is carried out in the presence of a base. Any suitable base that will deprotonate the hydrogen of the —XH moiety (for example, a phenol moiety when X is O) and allow the coupling may be used. Nonlimiting examples of bases are carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like.

The leaving group L is defined herein as any removable group customarily considered for chemical reactions, as will be known to the person skilled in the art. Suitable leaving groups are halogens, for example F, Cl, Br and I; alkyl sulfonate esters (—OSO₂R) wherein R is an alkyl group, for example methanesulfonate (mesylate), trifluoromethanesulfonate, ethanesulfonate, 2,2,2-trifluoroethanesulfonate, perfluoro butanesulfonate; aryl sulfonate esters (—OSO₂Ar) wherein Ar is an aryl group, for example p-toluoylsulfonate (tosylate), benzenesulphonate which may be unsubstituted or substituted by methyl, chlorine, bromine, nitro and the like; NO₃, NO₂, or sulfate, sulfite, phosphate, phosphite, carboxylate, imino ester, N₂ or carbamate.

The reaction may, in one embodiment, be conveniently carried out in a suitable inert solvent or diluent such as, for example, tetrahydrofuran, diethyl ether, aromatic amines such as pyridine; aliphatic and aromatic hydrocarbons such as benzene, toluene, and xylene; dimethylsulfoxide (DMSO), dimethylformamide (DMF), and dimethylacetamide (DMAC). The reaction is suitably carried out at a temperature in the range, for example, −20 to 120 C., for example at or near ambient temperature.

The coupling reagent defined hereinabove is a reagent capable of turning the carboxylic acid/thiocarboxylic acid of formula X into a reactive derivative thereof, thus enabling coupling with the respective amine amine to form an amide/thioamide bond. A suitable reactive derivative of a carboxylic acid/thiocarboxylic acid is, for example, an acyl halide/thioacyl halide, for example an acyl/thioacyl chloride formed by the reaction of the acid/thioacid and an inorganic acid chloride, for example thionyl chloride; a mixed anhydride, for example an anhydride formed by the reaction of the acid and a chloroformate such as isobutyl chloroformate; an active ester/thioester, for example an ester/thioester formed by the reaction of the acid/thioacid and a phenol, an ester/thioester or an alcohol such as methanol, ethanol, isopropanol, butanol or N-hydroxybenzotriazole; an acyl/thioacyl azide, for example an azide formed by the reaction of the acid/thioacid and azide such as diphenylphosphoryl azide; an acyl cyanide/thioacyl cyanide, for example a cyanide formed by the reaction of an acid and a cyanide such as diethylphosphoryl cyanide; or the product of the reaction of the acid/thioacid and a carbodiimide such as dicyclohexylcarbodiimide.

The reaction may be conveniently carried out in a suitable inert solvent or diluent as described hereinabove, suitably in the presence of a base such as triethylamine, and at a temperature in the range, as desribed above.

The compositions of this invention comprise a 5 alpha reductase inhbitor in combination with a SARM. In one embodiment, the 5 alpha reductase inhibitor is MK-906, a product of Merck, Sharp & Dohme (Mc Connell et al., J. Urol. 141: 239A, 1989). In another embodiment, the 5 alpha reductase inhibitor is 17β-N,N-diethylcarbamoyl-4-methyl-4-aza-5.alpha.-androstan-3-one (4-MA) (Brooks et al., Endocrinology 109: 830, 1981; Liang et al., Endocrinology 112: 1460, 1983). In another embodiment, the 5 alpha reductase inhibitor is a 4-azasteroid, which can be formed as in Liang et al., J. Biol. chem. 259: 734-739, 1984; and in Brooks et al., Steroids 47: 1-19, 1986.). In another embodiment, the 5 alpha reductase inhibitor is a 6-methylene-4-pregnene-3,20-dione, for example, as described (Petrow et al., J. Endocrinol. 95: 311-313, 1982). In another embodiment, the 5 alpha reductase inhibitor is a 4-methyl-3-oxo-4-aza-5.alpha.-pregnane-30(s) carboxylate (Kadohama et al., J. Natl. Cancer Inst. 74: 475-486, 1985).

As contemplated herein, the compositions of the present invention are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with ADIF, such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; g) decreasing the incidence of, halting or causing a regression of prostate cancer; and/or h) inducing apoptosis in a cancer cell.

As used herein, receptors for extracellular signaling molecules are collectively referred to as “cell signaling receptors”. Many cell signaling receptors are transmembrane proteins on a cell surface; when they bind an extracellular signaling molecule (i.e., a ligand), they become activated so as to generate a cascade of intracellular signals that alter the behavior of the cell. In contrast, in some cases, the receptors are inside the cell and the signaling ligand has to enter the cell to activate them; these signaling molecules therefore must be sufficiently small and hydrophobic to diffuse across the plasma membrane of the cell.

Steroid hormones are one example of small hydrophobic molecules that diffuse directly across the plasma membrane of target cells and bind to intracellular cell signaling receptors. These receptors are structurally related and constitute the intracellular receptor superfamily (or steroid-hormone receptor superfamily). Steroid hormone receptors include progesterone receptors, estrogen receptors, androgen receptors, glueocorticoid receptors, and mineralocorticoid receptors. In one embodiment, the present invention is directed to androgen receptors.

In addition to ligand binding to the receptors, the receptors can be blocked to prevent ligand binding. When a substance binds to a receptor, the three-dimensional structure of the substance fits into a space created by the three-dimensional structure of the receptor in a ball and socket configuration. The better the ball fits into the socket, the more tightly it is held. This phenomenon is called affinity. If the affinity of a substance is greater than the original hormone, it will compete with the hormone and bind the binding site more frequently. Once bound, signals may be sent through the receptor into the cells, causing the cell to respond in some fashion. This is called activation. On activation, the activated receptor then directly regulates the transcription of specific genes. But the substance and the receptor may have certain attributes, other than affinity, in order to activate the cell. Chemical bonds between atoms of the substance and the atoms of the receptors may form. In some cases, this leads to a change in the configuration of the receptor, which is enough to begin the activation process (called signal transduction).

In another embodiment, the present invention is directed to compositions comprising selective androgen receptor modulator compounds, which are antagonist compounds. A receptor agonist is a substance, which binds receptors and activates them. A receptor antagonist is a substance, which binds receptors and inactivates them. Thus, in one embodiment, the SARM compounds of the present invention are useful in binding to and inactivating steroidal hormone receptors. In one embodiment, the antagonist compound of the present invention is an antagonist which binds the androgen receptor. In another embodiment, the compound has high affinity for the androgen receptor.

Assays to determine whether the compounds of the present invention are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

An androgen receptor is an androgen receptor of any species, for example a mammal. In one embodiment, the androgen receptor is an androgen receptor of a human.

The SARM compounds utilized in the compositions of the present invention bind either reversibly or irreversibly to an androgen receptor. In one embodiment, the SARM compounds bind reversibly to an androgen receptor. In another embodiment, the SARM compounds bind reversibly to an androgen receptor of a mammal. In another embodiment, the SARM compounds bind reversibly to an androgen receptor of a human. Reversible binding of a compound to a receptor means that a compound can detach from the receptor after binding.

In another embodiment, the SARM compounds bind irreversibly to an androgen receptor. In one embodiment, the SARM compounds bind irreversibly to an androgen receptor of a mammal. In another embodiment, the SARM compounds bind irreversibly to an androgen receptor of a human. Thus, in one embodiment, the compounds of the present invention may contain a functional group (e.g. affinity label) that allows alkylation of the androgen receptor (i.e. covalent bond formation). Thus, in this case, the compounds are alkylating agents, which bind irreversibly to the receptor and, accordingly, cannot be displaced by a steroid, such as the endogenous ligands DHT and testosterone. An “alkylating agent” is defined herein as an agent, which alkylates (forms a covalent bond) with a cellular component, such as DNA, RNA or enzyme. It is a highly reactive chemical that introduces alkyl radicals into biologically active molecules and thereby prevents their proper functioning. The alkylating moiety is an electrophilic group that interacts with nucleophilic moieties in cellular components. For example, in one embodiment, an alkylating group is an isocyanate moiety, an electrophilic group which forms covalent bonds with nucleophilic groups (N, O, S etc.) in cellular components. In another embodiment, an alkylating group is an isothiocyanate moiety, another electrophilic group which forms covalent bonds with nucleophilic groups (N, O, S etc.) in cellular components. In another embodiment, an alkylating group is a haloalkyl (CH₂X wherein X is halogen), an electrophilic group which forms covalent bonds with nucleophilic groups in cellular components. In another embodiment, an alkylating group is a haloalkyl-amido (NHCOCH₂X wherein X is halogen), an electrophilic group which forms covalent bonds with nucleophilic groups in cellular components.

In one embodiment, the SARM compounds of the present invention are androgen receptor antagonists, which bind irreversibly to the androgen receptor of a mammal, for e.g. a human. In one embodiment, the compounds are alkylating agents.

The enzyme 5.alpha.-reductase catalyzes the conversion of testosterone to dihydrotestosterone (DHT), and an inhibitor of this enzyme prevents the conversion such that it selectively reduces DHT levels without reducing testosterone levels.

One of the principal mediators of androgenic activity in a target organ is 5.alpha.-dihydrotestosterone, which in many cases is a far more potent androgen than testosterone itself, and is formed locally in the target organ by the action of testosterone-5.alpha.-reductase. Inhibitors of testosterone-5.alpha.-reductase prevent or lessen symptoms of hyperandrogenic stimulation, and its combination with SARMs will, in one embodiment, serve to treat diseases, disorders and conditions which are stimulated, exacerbated or prolonged by elevated androgen production.

In one embodiment, the present invention provides a method of suppressing spermatogenesis in a subject, comprising the step of administering a composition of this invention to the subject, in an amount effective to suppress sperm production, in an amount effective to suppress sperm production.

In another embodiment, the present invention provides a method of contraception in a male subject, comprising the step of administering a composition of this invention to the subject, in an amount effective to suppress sperm production in the subject, thereby effecting contraception in the subject.

In another embodiment, the present invention further provides a method of hormone therapy, comprising the step of administering a composition of this invention to the subject, in an amount effective to effect a change in an androgen-dependent condition

In another embodiment, the present invention provides a method of hormone replacement therapy comprising administering a composition of this invention to the subject, in an amount effective to effect a change in an androgen-dependent condition

In another embodiment, the present invention further provides a method of treating a subject suffering from prostate cancer, comprising the step of administering a composition of this invention to the subject, in an amount effective to treat prostate cancer in the subject.

Androgen-dependent conditions, which may be treated according to the present invention include those conditions which are associated with aging, such as hypogonadism, sarcopenia, erythropoiesis, osteoporosis, and any other conditions later determined to be dependent upon low testosterone levels.

In another embodiment, the present invention further provides a method of treating a subject suffering from prostate cancer, comprising the step of administering to the subject the selective androgen receptor modulator compound of the present invention, and/or its analog, derivative, isomer, metabolite, pharmaceutically acceptable salt, pharmaceutical product, hydrate, N-oxide or any combination thereof, in an amount effective to treat prostate cancer in the subject.

In another embodiment, the present invention provides a method of suppressing, inhibiting or preventing prostate cancer, or its relapse in a subject, comprising the step of administering a composition of this invention to the subject, in an amount effective to suppress, inhibit or prevent prostate cancer or its relapse in the subject.

In another embodiment, the present invention further provides a method of preventing the recurrence of prostate cancer in a subject suffering from prostate cancer, comprising the step of administering a composition of this invention to the subject, in an amount effective to prevent the recurrence of prostate cancer in the subject.

In another embodiment, the present invention further provides a method of treating or preventing benign prostatic hyperplasia, comprising the step of administering a composition of this invention to the subject, in an amount effective to treat or prevent benign prostatic hyperplasia.

Furthermore, stimulation of the Androgen Receptor stimulates the production of tears, and thus the compositions of the present invention may be used to treat dry eye conditions. Therefore, according to another embodiment of the present invention, there is provided a method of treating or preventing a dry eye condition in a subject suffering from dry eyes, comprising the step of administering a composition of this invention to the subject, in an amount effective to treat or prevent dry eyes in the subject.

In another embodiment, the present invention provides a method of treating polycystic ovarian syndrome in a female subject, comprising the step of administering a composition of this invention to the subject, in an amount effective to treat polycystic ovarian syndrome.

In another embodiment, the present invention provides a method of suppressing, inhibiting, delaying onset or preventing diabetes, breast cancer, endometrial carcinoma or cardiovascular disease in a female subject suffereing from polycystic ovarian syndrome, comprising the step of administering a composition of this invention to the subject, in an amount effective to suppress, inhibit, delay onset, or prevent diabetes, breast cacner, endometrial carcinoma or cardiovascular disease in the subject.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission. As used herein, the term “delaying” means stopping, hindering, slowing down, postponing, holding up or setting back.

As used herin, the term “administering” refers to bringing a subject in contact with a SARM compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

The term “libido, as used herein, means sexual desire.

The term “erectile”, as used herein, means capable of being erected. An erectile tissue is a tissue, which is capable of being greatly dilated and made rigid by the distension of the numerous blood vessels which it contains.

“Hypogonadism” is a condition resulting from or characterised by abnormally decreased functional activity of the gonads, with retardation of growth and sexual development. “Osteopenia” refers to decreased calcification or density of bone. This is a term which encompasses all skeletal systems in which such a condition is noted.

“Osteoporosis” refers to a thinning of the bones with reduction in bone mass due to depletion of calcium and bone protein. Osteoporosis predisposes a person to fractures, which are often slow to heal and heal poorly. Unchecked osteoporosis can lead to changes in posture, physical abnormality, and decreased mobility.

“BPH (benign prostate hyperplasia)” is a nonmalignant enlargement of the prostate gland, and is the most common non-malignant proliferative abnormality found in any internal organ and the major cause of morbidity in the adult male. BPH occurs in over 75% of men over 50 years of age, reaching 88% prevalence by the ninth decade. BPH frequently results in a gradual squeezing of the portion of the urethra which traverses the prostate (prostatic urethra). This causes patients to experience a frequent urge to urinate because of incomplete emptying of the bladder and urgency of urination. The obstruction of urinary flow can also lead to a general lack of control over urination, including difficulty initiating urination when desired, as well as difficulty in preventing urinary flow because of the inability to empty urine from the bladder, a condition known as overflow urinary incontinence, which can lead to urinary obstruction and to urinary failure.

“Cognition” refers to the process of knowing, specifically the process of being aware, knowing, thinking, learning and judging. Cognition is related to the fields of psychology, linguistics, computer science, neuroscience, mathematics, ethology and philosophy. The term “mood” refers to a temper or state of the mind. As contemplated herein, alterations means any change for the positive or negative, in cognition and/or mood.

The term “depression” refers to an illness that involves the body, mood and thoughts, that affects the way a person eats, sleeps and the way one feels about oneself, and thinks about things. The signs and symptoms of depression include loss of interest in activities, loss of appetite or overeating, loss of emotional expression, an empty mood, feelings of hopelessness, pessimism, guilt or helplessness, social withdrawal, fatigue, sleep disturbances, trouble concentrating, remembering, or making decisions, restlessness, irritability, headaches, digestive disorders or chronic pain.

The term “hair loss”, medically known as alopecia, refers to baldness as in the very common type of male-pattern baldness. Baldness typically begins with patch hair loss on the scalp and sometimes progresses to complete baldness and even loss of body hair. Hair loss affects both males and females.

“Anemia” refers to the condition of having less than the normal number of red blood cells or less than the normal quantity of hemoglobin in the blood. The oxygen-carrying capacity of the blood is, therefore, decreased. Persons with anemia may feel tired and fatigue easily, appear pale, develop palpitations and become usually short of breath. Anemia is caused by four basic factors: a) hemorrhage (bleeding); b) hemolysis (excessive destruction of red blood cells); c) underproduction of red blood cells; and d) not enough normal hemoglobin. There are many forms of anemia, including aplastic anemia, benzene poisoning, Fanconi anemia, hemolytic disease of the newborn, hereditary spherocytosis, iron deficiency anemia, osteopetrosis, pernicious anemia, sickle cell disease, thalassemia, myelodysplastic syndrome, and a variety of bone marrow diseases. As contemplated herein, the SARM compounds of the present invention are useful in preventing and/or treating any one or more of the above-listed forms of anemia.

“Obesity” refers to the state of being well above one's normal weight. Traditionally, a person is considered to be obese if they are more than 20 percent over their ideal weight. Obesity has been more precisely defined by the National Institute of Health (NIH) as a Body to Mass Index (BMI) of 30 or above. Obesity is often multifactorial, based on both genetic and behavioral factors. Overweight due to obesity is a significant contributor to health problems. It increases the risk of developing a number of diseases including: Type 2 (adult-onset) diabetes; high blood pressure (hypertension); stroke (cerebrovascular accident or CVA); heart attack (myocardial infarction or MI); heart failure (congestive heart failure); cancer (certain forms such as cancer of the prostate and cancer of the colon and rectum); gallstones and gallbladder disease (cholecystitis); Gout and gouty arthritis; osteoarthritis (degenerative arthritis) of the knees, hips, and the lower back; sleep apnea (failure to breath normally during sleep, lowering blood oxygen); and Pickwickian syndrome (obesity, red face, underventilation and drowsiness). As contemplated herein, the term “obesity” includes any one of the above-listed obesity-related conditions and diseases. Thus the SARM compounds of the present invention are useful in preventing and/or treating obesity and any one or more of the above-listed obesity-related conditions and diseases.

“Prostate cancer” is one of the most frequently occurring cancers among men in the United States, with hundreds of thousands of new cases diagnosed each year. Over sixty percent of newly diagnosed cases of prostate cancer are found to be pathologically advanced, with no cure and a dismal prognosis. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer.

In one embodiment, the methods of the present invention comprise administering at least one 5 alpha reductase inhibitor (5-ARI) and at least one SARM compound as the active ingredients, however it is to be understood that multiple 5-ARI and SARM compounds may be utilized in the methods of this invention, and compositions comprising the same are to be considered as part of this invention. In another embodiment of this invention, the compositions and methods of use thereof may further comprise one or more therapeutic agents. These agents include, but are not limited to: LHRH analogs, reversible antiandrogens, antiestrogens, anticancer drugs, aromatase inhibitors, progestins, agents acting through other nuclear hormone receptors, selective estrogen receptor modulators (SERM), progesterone, estrogen, PDE5 inhibitors, apomorphine, bisphosphonate, sulfonurea compounds, statins or combinations thereof.

Thus, in one embodiment, the methods of the present invention comprise administering the selective androgen receptor modulator compound and 5-ARI, in combination with an LHRH analog, or in another embodiment, in combination with a reversible antiandrogen, or in another embodiment, with an antiestrogen, or in another embodiment, with an anticancer drug, or in another embodiment, in combination with an aromatase inhibitor, or in another embodiment, in combination with a progestin, or in another embodiment, in combination with an agent acting through other nuclear hormone receptors, or in another embodiment, in combination with a selective estrogen receptor modulators (SERM), or in another embodiment, in combination with a progesterone, or in another embodiment, in combination with an estrogen, or in another embodiment, in combination with a PDE5 inhibitor, or in another embodiment, in combination with apomorphine, or in another embodiment, in combination with a bisphosphonate.

In another embodiment, the compositions of this invention will comprise a suitable carrier or diluent.

In one embodiment, the compositions of this invention will comprise therapeutically effective amounts of the 5-ARI and SARM together with suitable diluents, preservatives, solubilizers, emulsifiers, adjuvant and/or carriers. A “therapeutically effective amount”, refers in one embodiment, to an amount, which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intravaginally, intracranially and intratumorally.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions of the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the composition can be delivered in a controlled release system. For example, the agents may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The compositions of this invention can comprise the active agents alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The compositions of this invention may be administered to a subject by, for example, subcutaneous implantation of a pellet; in a further embodiment, the pellet provides for controlled release of the active agents over a period of time. The composition may also be administered by intravenous, intraarterial, or intramuscular injection of a liquid preparation, oral administration of a liquid or solid preparation, or by topical application. Administration can also be accomplished by use of a rectal suppository or a urethral suppository.

The compositions of the invention may be prepared by known dissolving, mixing, granulating, or tablet-forming processes. For oral administration, the 5-ARI and SARM agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the 5-ARI and SARM agents or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of compositions, which contain active components is well understood in the art. Typically, such compositions are prepared as aerosols, which may be delivered to the nasopharynx, or as injectables, either as liquid solutions or suspensions; however, solid forms suitable for solution in, or suspension in, liquid prior to injection are also to be considered as part of this invention. The composition may also be emulsified. The active therapeutic ingredients may be mixed with excipients which are pharmaceutically acceptable and compatible with the active ingredients. Suitable excipients are, for example, water, saline, dextrose, glycerol, ethanol, or the like or any combination thereof.

In addition, the composition may contain minor amounts of auxiliary substances such as wetting or emulsifying agents, pH buffering agents which enhance the effectiveness of the active ingredient.

The active agents may be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium ammonium, calcium, or ferric hydroxides, and such organic bases as isopropylamine, trimethylamine, 2-ethylamino ethanol histidine, procaine, and the like.

For topical administration to body surfaces using, for example, creams, gels, drops, and the like, the SARM agents or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compounds may be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

Salts may be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

The following examples are presented in order to more fully illustrate the preferred embodiments of the invention. They should in no way be construed, however, as limiting the broad scope of the invention.

### EXPERIMENTAL DETAILS SECTION

### Example 1

### Pharmaceutical Compositions

The active ingredient is Formula II (>99.9% pure S-isomer). The inactive ingredients are lactose monohydrate, lactose fast-flo 316, Avicel PH102 (microcrystalline cellulose), magnesium stearate and colloidal silicon dioxide. The blended active and inactive ingredients are filled into white opaque hard gelatin capsules (size one).

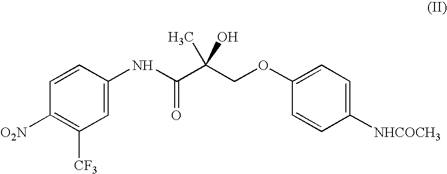

Quantitative Composition

TABLE 1
1 mg FORMULATION

| | | | Weight/Count | |
|---|---|---|---|---|
| | | Excipient | Per dosage | Weight/Count |
| Ingredient: | Manufacturer: | Purpose: | unit: | Per Batch*: |
| Formula II | ChemSyn | Active | 1.00 mg | 0.500 g |
| | Laboratories | | | |
| Lactose Monohydrate, | Foremost | Diluent/Filler | 80.00 mg | 40.000 g |
| NF | | | | |
| (#310 Regular) | | | | |
| Lactose Monohydrate, | Foremost | Filler/Flow-Aid | 196.45 mg | 98.225 g |
| NF | | | | |
| (#316 Fast-Flo | | | | |
| Modified, Spray-Dried) | | | | |
| Microcrystalline | FMC | Filler/Disintegrant | 30.00 mg | 15.000 g |
| Cellulose, NF (Avicel | | | | |
| PH102) | | | | |
| Silicon Dioxide, | Cabot | Flow-Aid | 1.00 mg | 0.500 g |
| Colloidal, USP/NF | | | | |
| (Cab-O-Sil M-5P) | | | | |
| Magnesium Stearate, | Mallinckrodt | Lubricant | 1.55 mg | 0.775 g |
| NF | | | | |
| HyQual | | | | |
| Capsule, Hard Gelatin | Capsugel | Capsule | 1 (Count) | 500 (Count) |
| Size 1, White Opaque | | | | |

*Batch size based on 500 capsules but may change depending on requirements

TABLE 2
0.1 mg FORMULATION

| | | | Weight/Count | |
|---|---|---|---|---|
| | | Excipient | Per dosage | Weight/Count |
| Ingredient: | Manufacturer: | Purpose: | unit: | Per Batch*: |
| Formula II | ChemSyn | Active | 0.10 mg | 0.050 g |
| | Laboratories | | | |
| Lactose Monohydrate, | Foremost | Diluent/Filler | 80.00 mg | 40.000 g |
| NF | | | | |
| (#310 Regular) | | | | |
| Lactose Monohydrate, | Foremost | Filler/Flow-Aid | 197.35 mg | 98.675 g |
| NF | | | | |
| (#316 Fast-Flo | | | | |
| Modified, Spray-Dried) | | | | |
| Microcrystalline | FMC | Filler/Disintegrant | 30.00 mg | 15.000 g |
| Cellulose, NF (Avicel | | | | |
| PH102) | | | | |
| Silicon Dioxide, | Cabot | Flow-Aid | 1.00 mg | 0.500 g |
| Colloidal, USP/NF | | | | |
| (Cab-O-Sil M-5P) | | | | |
| Magnesium Stearate, | Mallinckrodt | Lubricant | 1.55 mg | 0.775 g |
| NF | | | | |
| HyQual | | | | |
| Capsule, Hard Gelatin | Capsugel | Capsule | 1 (Count) | 500 (Count) |
| Size 1, White Opaque | | | | |

*Batch size based on 500 capsules but may change depending on requirements

Specifications and Analytical Methods for Inactive Compounds

All inactive ingredients included in the formulation have monographs that denote full compendial testing per Standard Operating Procedure of Metrics, Inc. (1240 Sugg Parkway Greenville, N.C. 27834) with the exception of the active ingredient and the capsule shell. Active pharmaceutical ingredient and capsule shells are provided with Certificate of Analysis.

Method of Manufacturing

Capsules of Formula II are manufactured using the formulations as set forth in Table 1 (1 mg formulation) and Table 2 (0.1 mg formulation).

For 1 mg Formula II capsules: the indicated amount of active and inactive ingredients are dispensed. 0.5 grams of Formula II (active pharmaceutical ingredient, API) are diluted by placing API and an equal part of lactose monohydrate (0.5 grams) in mortar. The mixture is ground with a pestle until homogenous. The mixture is diluted again by adding one additional gram of lactose monohydrate to the mixture and grinding until homogenous. The diluted active:lactose monohydrate mixture is blended with 38.5 grams of lactose monohydrate, 98.225 grams of lactose fast-flo, and 15 grams of Avicel PH102 in a one pint V-shell blender for 15 minutes. Approximately 10 grams of the blend is removed and added to 0.5 grams of Cab-O-Sil. The mixture is mixed with a spatula and screened through a 20-mesh screen. 0.775 grams of magnesium stearate are independently screened through a 20-mesh screen. The screened ingredients (10 grams of initial blend with Cab-O-Sil, and magnesium stearate) are added to the remainder of the initial blend in the one pint V-shell blender. All ingredients are blended together in a V-shell blender for five minutes. Capsule shells (500 count) are dispensed into a Chemipharm Manual Capsule Filler. 31 grams of blended mixture are manually filled into 100 capsules using the Chemipharm Manual Capsule Filler. The capsules are manually packaged and labeled. Each capsule contains 1 milligram of active and 309 milligrams of inactive ingredients.

For 0.1 mg Formula II capsules: the same Method of Manufacturing is used, the amounts of Formula II API and inactive ingredients are adjusted accordingly (Table 2).

### Example 2

### Additional Nonsteroidal Ligands with Androgenic and Anabolic Activity Synthetic Procedures

(2R)-1-Methacryloylpyrrolidin-2-carboxylic Acid (R-129). D-Proline (R-128, 14.93 g, 0.13 mol) was dissolved in 71 mL of 2 N NaOH and cooled in an ice bath; the resulting alkaline solution was diluted with acetone (71 mL). An acetone solution (71 mL) of metacryloly chloride 127 (13.56 g, 0.13 mol) and 2N NaOH solution (71 mL) were simultaneously added over 40 min to the aqueous solution of D-proline in an ice bath. The pH of the mixture was kept at 10-11° C. during the addition of the metacryloly chloride. After stirring (3 h, room temperature), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄ filtered through Celite, and evaporated in vacuo to give the crude product as a colorless oil. Recrystallization of the oil from ethyl ether and hexanes afforded 16.2 (68%) of the desired compound as colorless crystals: mp 102-103° C. (lit. [214] mp 102.5-103.5° C.); the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral canter), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd for C₉H₁₃NO₃: C 59.00, H 7.15, N 7.65. Found: C, 59.13; H, 7.19; N, 7.61.

(3R,8aR)-3-Bromomethyl-3-methyl-tetrahydro-pyrrolo[2,1c][1,4]oxazine-1,4-dione (R, R-130). A solution of NBS (23.5 g, 0.132 mol) in 100 mL of DMF was added dropwise to a stirred solution of compound R-129 (16.1 g, 88 mmol) in 70 mL of DMF under argon at room temperature, and the resulting mixture was stirred 3 days. The solvent was removed in vacuo, and a yellow solid was precipitated. The solid was suspended in water, stirred overnight at room temperature, filtered, and dried to give 18.6 (81%) (smaller weight when dried ˜34%) of the title compound as a yellow solid: mp 152-154° C. (lit. [214] mp 107-109° C. for the S-isomer); ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24; H, 4.61; N, 5.34. Found: C, 41.46; H, 4.64; N, 5.32.

(2R)₃-Bromo-2-hydroxy-2-methylpropanoic Acid (R-131). A mixture of bromolactone R-130 (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite, and evaporated in vacuo to dryness. Recrystallization from toluene afforded 10.2 g (86%) of the desired compound as colorless crystals: mp 107-109° C. (lit. [214] mp 109-113° C. for the S-isomer); ¹H NMR (300 MHz, DMSO-d₄) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5, (c=2.6, MeOH); Anal. Calcd for C₄H₇BrO₃: C 26.25, H 3.86. Found: C 26.28, H 3.75.

N-[4-Nitro-3-(trifluoromethyl)phenyl]-(2R)-3-bromo-2-hydroxy-2-methylpropanamide (R-132). Thionyl chloride (8.6 g, 72 mmol) was added dropwise under argon to a solution of bromoacid R-131 (11.0 g, 60 mmol) in 70 mL of DMA at −to −10° C. The resulting mixture was stirred for 2 h under the same conditions. A solution of 4-nitro-3-trifluoromethyl-aniline (12.4 g, 60 mmol) in 80 mL of DMA was added dropwise to the above solution, and the resulting mixture was stirred overnight at room temperature. The solvent was removed on Rotavapor using high vacuum oil pump; the residue was diluted with saturated NaHCO₃ solution, and extracted with ethyl ether (100 mL×3). Combined extracts were dried over anhydrous Na₂SO₄, filtered through Celite, and purified by flash chromatography on silica gel, using methylene chloride as eluent to afford 18.0 g (80%) of the desired compound: mp 98-100° C. (R_{f}=0.2, silica gel, CH₂Cl₂); ¹H NMR (300 MHz, DMSO-d₆) δ 10.54 (s, 1H, NH), 8.54 (d, J=2.1 Hz, 1H, ArH), 8.34 (dd, J=9.0 Hz, J=2.1 Hz, 1H, ArH), 8.18 (d, J=9.0 Hz, 1H, ArH), 6.37 (s, 1H, OH), 3.82 (d, J=10.4 Hz, 1H, CHHₐ), 3.58 (d, J=10.4 Hz, 1H, CHH_{b}), 1.48 (s, 3H, Me); ¹³C NMR (75 MHz, DMSO-d₆) δ 173.6 (C═O), 143.0, 127.2, 123.2, 122.6 (q, J=33.0 Hz), 122.0 (q, J=271.5 Hz), 118.3 (q, J=6.0 Hz), 74.4, 41.4, 24.9; IR (KBr) 3344 (OH), 1680 (C═O), 1599, 1548 (C═C, Ar), 1427, 1363, 1161 cm⁻¹; MS (ESI): m/z 370.8 (M)⁺; Anal. Calcd. for C₁₁H₁₀BrN₂O₄: C, 35.60; H, 2.72; N, 7.55. Found: C, 35.68; H, 2.72; N, 7.49.

N-[4-nitro-3-trifluoromethyl)phenyl]2S)-3-[4(acetylamino)phen oxy]-2-hydroxy-2-methylpropanamide (S-147, Compound IV). The title compound was prepared from compound R-132 (0.37 g, 1.0 mmol), 4-acetamidophenol (0.23 g, 1.5 mmol) K₂CO₃ (0.28 g, 2.0 mmol), and 10% of benzyltributylammonium chloride as a phase transfer catalyst in 20 mL of methyl ethyl ketone was heated at reflux overnight under argon. The reaction was followed by TLC, the resulting mixture was filtered through Celite, and concentrated in vacuo to dryness. Purification by flash column chromatography on silica gel (hexanes-ethyl acetate, 3:1) yielded 0.38 g (86%) (R_{f}=0.18 hexanes-ethyl acetate, 3:1) of the desired compound as a light yellow powder: mp 70-74° C.; The solid can be recrystalized from ethyl acetate and hexane); ¹H NMR (300 MHz, DMSO-d₆) δ 10.62 (s, 1H, NH), 9.75 (s, 1H, NH), 8.56 (d, J=1.9 Hz, 1H, ArH), 8.36 (dd, J=9.1 Hz, J=1.9 Hz, 1H, ArH), 8.18 (d, J=9.1 Hz, 1H, ArH), 7.45-7.42 (m, 2H, ArH), 6.85-6.82 (m, 2H, ArH), 6.25 (s, 1H, OH), 4.17 (d, J=9.5 Hz, 1H, CHHₐ), 3.94 (d, J=9.5 Hz, 1H, CHH_{b}), 1.98 (s, 3H, Me), 1.43 (s, 3H, Me); ¹³C NMR (75 MHz, DMSO-d₆) δ 174.6 (C═O), 167.7, 154.2, 143.3, 141.6, 132.8, 127.4, 123.0, 122.7 (q, J=33.0 Hz), 122.1 (q, J=271.5 Hz), 120.1, 118.3 (q, J=6.0 Hz), 114.6, 74.9, 73.8, 23.8, 23.0; IR (KBr) 3364 (OH), 1668 (C—O), 1599, 1512 (C═C, Ar), 1457, 1415, 1351, 1323, 1239, 1150 1046 cm⁻¹; MS (ESI): m/z 464.1 (M+Na)⁺; Anal. Calcd. for C₁₉H₁₈F₃N₃O₆: C, 51.71; H, 4.11; N, 9.52. Found: C 52.33, H 4.40, N 9.01.

The synthesis of the various ether analogs of Compound IV, such as, but not limited to, compounds I-III and V-VII provided herein, utilizes the common intermediate that is the final reaction step. Bromo-intermediates are used which allow various phenolic compounds to displace the bromide to give the desired ether product. Bromohydrin was converted to an epoxide and to open the epoxide to give the same desired ether product.

### Example 3

### 5-ARI and SARM Compositions

A Tablet formulation, with scored tablets for oral use, may be prepared containing, in one embodiment, 500 mg. of each active ingredient, or in another embodiment, 250 mg of each active ingredient. The tablets may be prepared, in one embodiment, from the following ingredients:

| | Gm. |
|---|---|
| 17β-N,N--diethylcarbamoyl-4-methyl- | 5000 |
| 4-aza-5.alpha.-androstan-3-one | |
| Formula II | 5000 |
| Starch, U.S.P. | 350 |
| Talc, U.S.P. | 250 |
| Calcium stearate | 35 |

The active ingredients are granulated with a 4% w./v. aqueous solution of methylcellulose U.S.P. (1500 cps). To the dried granules is added a mixture of the remainder of the ingredients and the final mixture compressed into tablets of proper weight.

Capsules—hard gelatin capsules for oral use, each containing 250 mg. of active ingredients may be prepared, in another embodiment from the following ingredients:

| | Gm |
|---|---|
| 17β-N,N--diethylcarbamoyl-4-methyl- | 2500 |
| 4-aza-5.alpha.-androstan-3-one | |
| Formula II | 2500 |
| Lactose, U.S.P. | 1000 |
| Starch, U.S.P. | 300 |
| Talc, U.S.P. | 65 |
| Calcium Stearate | 25 |

The active ingredients are mixed with the starch lactose mixture followed by the talc and calcium stearate. The final mixture is then encapsulated in the usual manner. Capsules containing 0.1, 1, 5, 10, 15, 25, 50, and 100 mg. of each active ingredient is also prepared by substituting 1, 10, 50, 100, 150, 250, 500, and 1000 gm. of 2500 gm. in the above formulation. In another embodiment, the concentration of the SARM is 10, or in another embodiment 25, or in another embodiment 50% that of the 5-AR₁, in any composition of this invention.

Soft elastic capsules—One-piece soft elastic capsules for oral use, each containing 500 mg. of each, or 250 mg of each active material are prepared in the usual manner by first dispersing the active material in sufficient corn oil to render the material capsulatable.

Aqueous suspension—An aqueous suspension for oral use containing in each 5 ml., 0.25 g. of each active ingredient is prepared from the following ingredients:

| | Gm. |
|---|---|
| 17β-N,N--diethylcarbamoyl-4-methyl- | 500 |
| 4-aza-5.alpha.-androstan-3-one | |
| Formula II | 500 |
| Methylparaben, U.S.P. | 7.5 |
| Propylparaben, U.S.P. | 2.5 |
| Saccharin sodium | 12.5 |
| Glycerin | 3000 |
| Tragacanth powder | 10 |
| Orange oil flavor | 10 |
| F.D. & C. orange dye | 7.5 |
| Deionized water, q.s. to 10,000 ml | |

It will be appreciated by a person skilled in the art that the present invention is not limited by what has been particularly shown and described hereinabove. Rather, the scope of the invention is defined by the claims that follow:

1.-58. (canceled)

59. A composition comprising a 5-alpha reductase inhibitor and a selective androgen receptor modulator (SARM) compound represented by the structure of formula III: 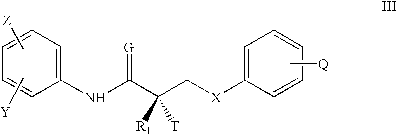
wherein
X is a bond, O, CH₂, NH, S, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is CN SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

60. The composition according to claim 59, wherein said selective androgen receptor modulator compound is an analog, isomer, metabolite, derivative, pharmaceutically acceptable salt, pharmaceutical product, N-oxide, hydrate or any combination thereof of said compound.

61. The composition according to claim 59, wherein G is O.

62. The composition according to claim 59, wherein T is OH.

63. The composition according to claim 59, wherein R₁ is CH₃.

64. The composition according to claim 59, wherein X is O.

65. The composition according to claim 59, wherein Z is NO₂.

66. The composition according to claim 59, wherein Z is CN.

67. The composition according to claim 59, wherein Y is CF₃.

68. The composition according to claim 59, wherein O is CN.

69. The composition according to claim 59, wherein G is O, T is OH, R₁ is CH₃, X is O, Z is NO₂, Y is CF₃, and Q is CN.

70. The composition according to claim 59, wherein said compound is an androgen receptor antagonist.

71. The composition according to claim 59, wherein said compounds binds irreversibly to an androgen receptor.

72. The composition according to claim 59, wherein said compound is an androgen receptor antagonist which binds irreversibly to an androgen receptor.

73. The composition according to claim 59, represented by the structure of formula V:

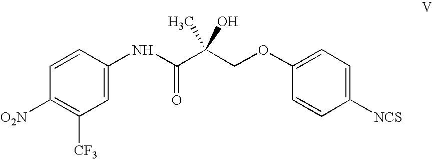

74. The composition according to claim 59, wherein said 5-alpha reductase inhibitor is dutasteride or finasteride, or a combination thereof.

75. The composition according to claim 74, wherin said carrier or diluent is lactose monohydrate, microcrystalline cellulose, or a mixture thereof.

76. The composition according to claim 59, further comprising a lubricant.

77. The composition according to claim 75, wherein said lubricant is magnesium stearate.

78. The composition according to claim 59, further comprising a flow-aid.

79. The composition according to claim 77, wherein said flow aid is colloidal silicon dioxide.

80. The composition according to claim 59, further comprising one or more additives selected from a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

81. The composition according to claim 59, wherein said composition is in the form of a pellet, a tablet, a capsule, a solution, a suspension a dispersion, an emulsion, an elixir, a gel, an ointment, a cream, or a suppository.

82. The composition according to claim 59, wherein said composition is in the form of a capsule.

83. The composition according to claim 59, wherein said composition is in a form suitable for oral, intravenous, intraarterial, intramuscular, subcutaneous, parenteral, transmucosal, transdermal, or topical administration.

84. The composition according to claim 59, wherein said composition is in a form suitable for oral administration.

85. The composition according to claim 59, wherein said composition is a controlled release composition.

86. The composition according to claim 59, wherein said composition is an immediate release composition.

87. The composition according to claim 59, wherein said composition is a liquid dosage form.

88. The composition according to claim 59, wherein said composition is a solid dosage form.

89.-93. (canceled)

93. A method of treating prostate cancer in a subject, comprising the step of administering to said subject the composition of claim 59, in an amount effective to treat prostate cancer in said subject.

94. A method of treating the recurrence of prostate cancer in a subject suffering from prostate cancer, comprising the step of administering to said subject the composition of claim 59, in an amount effective to treat the recurrence of prostate cancer in said subject.

95. A method of treating benign prostatic hyperplasia in a subject, comprising the step of administering to said subject the composition of claim 59, in an amount effective to treat benign prostatic hyperplasia in said subject.

96. A method of inhibiting, suppressing or preventing benign prostatic hyperplasia in a subject, comprising the step of administering to said subject the composition of claim 59, in an amount effective to inhibit, suppress or prevent benign prostatic hyperplasia in said subject.

97. A method of treating a subject having a hormone related condition, comprising the step of administering to said subject the composition of claim 59, in an amount effective to effect a change in an androgen-dependent condition.

98-99. (canceled)

100. A method f treating polycystic ovarian syndrome in a female subject, comprising the step of administering to said subject the composition of claim 59, in an amount effective to treat polycystic ovarian disease in the subject.

101. A methods of suppressing, inhibiting, delaying onset or preventing diabetes, breast cancer, endometrial carcinoma or cardiovascular disease in a female subject suffereing from polycystic ovarian syndrome, comprising the step of administering to said subject the composition of claim 59, in an amount effective to suppress, inhibit, delay onset, or prevent diabetes, breast cacner, endometrial carcinoma or cardiovascular disease in the subject.
